(19) United States
(12) Patent Application Publication
(10) Pub. No.: US 2017/0206333 A1
(43) Pub. Date: Jul. 20, 2017

(54) Indicator Incorporating Complex Properties of Systems

(71) Applicant: Calin Pop, Brooksvile, FL (US)

(72) Inventor: Calin Pop, Brooksvile, FL (US)

(21) Appl. No.: 15/404,504

(22) Filed: Jan. 12, 2017

Related U.S. Application Data
(63) Continuation of application No. 62/278,530, filed on Jan. 14, 2016.

(51) Int. Cl. G06F 19/00; G01N 33/49

(52) U.S. Cl. CPC G06F 19/3431; G06F 19/322; G01N 33/49

(57) ABSTRACT

A highly customizable indicator based on a mathematical processing of two or more parameters of a particular system therefore describing more complex properties of the system. The indicator can be used primarily in health care and related fields including risk evaluation and stratification, but can be used in any kind of human endeavor including, but not limited to humanistic or scientific fields, commerce, trade, management, insurance, or other purposes.

### BACKGROUND OF THE INVENTION

Field of the Invention

This invention relates to a novel indicator that can be used for many purposes and in many different ways in different circumstances and life settings, but especially in health care related fields for example health risk evaluation and stratification.

In other aspects it relates to other humanistic or scientific fields that can use a customized version of this indicator. Novel and specially customized versions of our indicator can describe in one simple number highly complex properties and characteristics of materials, people, behaviors, etc. The indicators can be used to predict future parameters, behaviors, risk factors, and others.

Description of Related Art

For many years, in various fields of human endeavor, the methods of quantifying complex systems are rudimentary at best. Some specific fields have some rough and approximate indicators like, for example, hemoglobin A1c in diabetes care, the Mini-Mental Status Examination score in Alzheimer's or the Consumer Report's score on my car reliability. With the help of our more precise indicator we could evaluate complex systems in a much more accurate and/or customized way.

For example we strongly believe that many of us would like to evaluate diabetes by much more than just measuring the hemoglobin A1c marker in the blood. For example, we can easily add to an index describing diabetes other important parameters related to this illness such as fructosamine, creatinine or another kidney function indicator, Body Mass Index, triglycerides and any other parameter we may deem appropriate and relevant for a condition like diabetes. A final diabetes indicator, constructed based on the weighted contribution of all the above parameters, shall be one final number incorporating all of the above diabetic parameters in the specific proportion and weight to the index we deem appropriate.

The factors that may enter as parameters into the indicator can be either factors generally well documented by previous research like the Hemoglobin A1c marker for diabetes or factors that can be determined by us, thus including in the final indicator one or more elements we believe are important, but may not be well documented by previous research. Parameters we may introduce in the formula may include, for example, results of a self-evaluating depression scale that we may have just designed, or can be any combinations of known and/or unknown parameters we desire. The addition and contribution of know and/or unknown parameters to the indicator is a clear advantage, as it will make the indicator much more customized for any specific field, and much more sensitive and specific than present day approximations or rough indicators are currently.

Prior art research yields many health indicators or general indicators as related to technical fields, most of them involving a physical machinery or apparatus. In contrast, our indicator consists of a specific, customized mathematical formula that yields a very specific number called an indicator.

Since most of the previous research was geared towards technical aspects of a particular system or machinery, most of the indicators discovered in prior art do not apply to our type of indicator.

We believe that our indicator, even though it is sometimes derived from the mathematical computations of known parameters that may be widely available, it becomes an element of crucial significance in the evaluation and monitoring of a complex system. Optimizing the indicator to the exact needs of the user can be of tremendous importance in evaluating, monitoring, stratifying risk, or treating a multitude of common conditions including but not limited to the aging process and chronic diseases that affect humanity.

Our invention consists in using much more than one standard or classic parameters, in various proportion and weight assignment to describe a complex condition. The more parameters we use in the correct combination and correct weight assignment the more accurate the indicator will be compared to the standard methods of assessing a particular complex system, if any said standard methods even exist.

The design and application in health care of a single, individual, indicators of a complex system has been previously described, for example the Hemoglobin A1c marker in diabetes. The novelty in our invention is the intent to combine together several parameters, in different proportion, weight assignment and applying other possible filters to yield a true novel indicator with a better reliability and specificity for any specific application, than any individual marker alone can yield.

Another novelty of our approach in the field of complex systems and indicators is the flexibility of customization in a particular field.

Another novelty of our approach is the ability to apply the indicator to the initial evaluation, ongoing intervention and post intervention phase of any intervention of any kind, upon a complex system. Our approach can also be used in determining historical progression of indexes when older, previous parameters are still available.

In previous art there is no mention of the intention or attempt to extensively maximize, calibrate or control the indicator relative to the process involved. We provide methods of maximizing the sensitivity and specificity of our indicator in such a way as to produce the maximum reliability for any particular field.

We also explain why our indicator has practically an unlimited number of potential uses in different types of human activity, evaluation, monitoring, treatment, and others.

While not all existing parameters we may choose have the same importance in establishing an appropriate indicators, we can adjust this by giving different parameters a different weight when participating to the construction of a specific indicator.

The approach described in U.S. patent application Ser. No. 09/852,689 titled “Health indicator measuring device” filing date 2001 May 11 reveals a health indicator measuring device which includes an arithmetical operation means for computing data of a lean body per body height or a data of a body fat per body height on the basis of the respective data of body weight, body fat and body height. In other words this is a device that derives a health indicator based on specific measurements of body weight, height and fat content.

U.S. patent application Ser. No. 14/565,860 titled “Health risk indicator determination” filing date: 2014 Dec. 10 also describes a health indicator measuring device which includes an arithmetical operation means for computing data of a lean body per body height or a data of a body fat per body height on the basis of the respective data of body weight, body fat and body height. It involves a wearable heart rate monitor and the health determination is rather limited to a very small range of body parameters and heart rhythm determinations.

On the contrary, our health indicator comprises the results of several laboratory blood tests measuring different functions of different body systems, as well as also having the option to add on some physical body measurements as parameters to the formula if they arc deemed important.

Laboratory blood testing and their different results are therefore crucial in determining the value of our health indicator. The addition of body fat composition, height weight or any other physical body measurements is only optional, not crucial, and may only help in a limited way, by fine tuning our indicator. Our indicator does not depend on any specific device or apparatus.

The U.S. patent application Ser. No. 09/560,996 titled “Measuring user health as measured by multiple diverse health measurement devices utilizing a personal storage device” filing date: 2000 Apr. 28 relates in general to a health monitoring device and in particular to a method, system and program for monitoring the outputs of multiple diverse health measurement devices. Still more particularly, the present invention relates to a method, system and program for monitoring physical health indicators received from multiple diverse health measurement devices at a single personal storage device and managing multiple diverse health control devices according to normal physical health levels for a user retrieved from the personal storage device.

This particular patent application describes an apparatus, a device that monitors input from other devices, usually a recording of some physical parameters only. Their application is about a physical device recording some very limited physical measurements.

On the contrary our health index uses no device, and computes several biochemical markers. Our health index is based in a high proportion on blood bio chemistry and laboratory test results. Our indicator has no required physical device as a required instrument as a central piece of the health index construction.

The patents, applications, journals and scholar articles so far describe prior art that uses simple, single feature indicators mostly in the technical fields to evaluate the composition of a material or the reliability of a technical component. The vast majority of indicators presently used are clearly relating to very limited and narrow fields of use, mostly technical in scope.

On the contrary, our novel indicator can be used for many purposes and in many different ways in many different circumstances and life settings, and especially in health care related fields. The variety of scope, reliability and flexibility of our indicator are unique.

### SUMMARY OF THE INVENTION

The present patent application describes a novel indicator that is able to accurately quantify conditions, states, properties or characteristics that were previously difficult or impossible to measure or accurately quantify. The indicator is derived from a plurality of parameters or values, for example laboratory blood tests results, chosen in direct relation with the purpose of the indicator.

The parameters values are introduced in a mathematical formula and the result of the mathematical formula computations shall be a unique number, or an assigned qualifier, like a letter or other means to of classifying information. The indicator finally describes the complexity of the parameters relative to our criteria. The indicator also describes the parameters interactions towards the feature, quality, or characteristic described by the indicator, in a way we desire to.

This invention can find utility in a broad range of applications, including medical applications. One example of use in health care is for health risk evaluation and stratification. Previous uses of patented indicators were not flexible and were in general severely limited only to extremely restricted and very narrow single scope, individual, utility applications like a time indicator, thaw indicator, vacuum indicator or sterility indicator.

One advantage of our indicator is that it quickly and accurately gives an instant overview, a valuable glimpse, into how a complicated and difficult to approximate or to quantify condition presents itself. In other words our indicator may quantify in one quick number, conditions that were previously difficult to quantify, like fatigue, aging, memory, risk of diabetes complications, as well as suitability of a specific candidate for a position or food suitability for consumption.

Another advantage is derived from the fact that the indicator is very flexible, being able to easily be adjusted and applied to various applications in many different fields of science, commerce, humanistic fields or even human or non-human behavior. The indicator can be applied to any field where a certain indicator is desired to quantify and characterize a combination or interaction between two or more quantified, specific, measurable, or just given or self-determined component.

Other advantages derive from the fact that the indicator can be easily adjusted to reflect different parameters in different ways, therefore allowing the indicator to be more or less sensitive and/or specific to a certain parameter and to be able to be more or less influenced by a certain parameter. This property is useful in designing indicators with a specific and desired range of normal and abnormal values and a different ability of becoming more abnormal earlier with only minimal variations of the parameters of input beyond the abnormality point.

Other advantages derive from the fact that complex conditions can be evaluated and monitored over time.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other indicators for carrying out the same purposes of the present invention and that such other structures do not depart from the spirit and scope of the invention in its broadest form.

### BRIEF DESCRIPTION OF THE DRAWINGS

This application contains no drawings. For a better illustration of our indicator concept we attach a depiction of the control panel table containing one particular example of our indicator for a specialized field, where five parameters (x,y,z,q,r) are leading to a final indicator number, in this case 107.6, which is slightly abnormal.

As can be depicted from the table, each of the parameters are given specific descriptors like higher and lower limits, a median value, an abnormality point, a direction, where higher is better versus higher is worse, and an abnormality descriptor like linear versus logarithmic. These descriptors are highly customizable and variable in order to customize the indicator for various applications. Other descriptors of the parameters can be added to the formula depending of the type of indicator we need to design.

Generally, the normal values of indicator are depicted in green and abnormal values are depicted in red; however, colors, placement, design and style of indicator may vary.

Ultimately, all that counts for the characterization of the complex system behavior, is the indicator, depicted in our example in the upper right corner.

It is important to understand that the illustration of the control panel table is not necessary for the utilization of an index. The control panel table and its' settings are very important in the initial stages when the indicator is being designed, fine-tuned and customized. After the indicator is customized, the table may only work in the background, and the user may only see the fields to input the parameters, in this case the field named “Values” and the final indicator number.

Another important display option is to show the abnormal parameter values and their possible weighted contribution to the final indicator which is especially important when the final indicator comes out abnormal.

### DETAILED DESCRIPTION OF THE INVENTION

Our described indicator is built upon several components named parameters which when plugged into a mathematical formula generate a final indicator number.

The mathematical formula behind the indicator takes into account several assigned parameters, generally two or more parameters. Depending on the assigned parameters and parameters' real value on input when compared with the normal values of parameters, the mathematical formula will create an indicator number.

Our novel indicator therefore starts with considering, then utilizing, several parameters that are used as input in order to build a customized, specific indicator.

Depending on the field we want to use the customized indicator, the input parameters will be different. Parameters can be added, subtracted, minimized, amplified or maximized depending upon the particular requirement of an indicator. In other words the impact of Hemoglobin A1c in diabetes will be amplified in a diabetes indicator because it is more important than, say, triglycerides. Triglycerides are considered a normal, non-amplified parameter. Another possible parameter, like for example the blood sugar range for the last three days will be minimized compared to other parameters, because its' assigned lesser importance.

In one example a diabetes indicator may contain Hemoglobin A1c, blood sugar daily range, blood sugar incremental raise pre and post meals, a kidney function number like creatinine or cystatin C, triglycerides, fructosamine, blood sugar range for the last three days, etc.

In another example a fatigue index can contain parameters such as the following but not limited to these parameters: the level of hemoglobin, sex hormones like testosterone, a depression self-evaluation score, a toxicity index, and a ratio of two or three types of thyroid hormones.

The indicator has an assigned normal range and an out of normal range. The further away the indicator number is from the normal values, the worse a specific condition would be.

In one example, the normal ranges can be below a number, in other examples, the normal ranges can be established above a number. The number changing from normal to abnormal or the abnormality direction can be changed depending upon the customization level required. In one example an indicator above 100 is abnormal and in another example an indicator below 100 is abnormal. The ranges of any particular indicator can be initially established or modified. In one example the indicator is ranging from 20 to 500 and the inflexion point of abnormality is, in this example, the number 100, or whatever number between the two extreme ends of the set up range we determine in advance.

In one example, illustrated in the table attached to this application, the indicator value of less than 100 is considered normal and an indicator value of more than 100 is considered abnormal. The indicator becomes more and more abnormal the further from the inflection abnormality point it is. For example if the range of abnormality is from 101, barely abnormal, to 500 the worst, then an indicator of 400 is worse than an indicator of 200.

In this particular example the higher the indicator number above 100, the more abnormal the indicator is. The abnormality point for the indicator in the above example is 100, however any number can be chosen to be the abnormality point.

In another example a range of indicators between 1 and 20 can be set up with an abnormality point below 10, the higher the number the better.

As a particular example, a general health indicator would be created by the mathematical formula, by taking two or more laboratory values or components out of real laboratory tests.

The parameters set up for the above real values of laboratory testing, are previously designed to be attributed with a normal range, with higher and lower limits, a median value range, an abnormality point, a direction, where higher is better versus higher is worse, and an abnormality descriptor such as linear versus logarithmic. Some laboratory test results that are slightly abnormal are extremely important and may have a great impact on the indicator, while other laboratory test results are less important and may have only a minimal impact on the indicator.

The differentiated impact of different laboratory test results or any parameters, is accomplished by assigning each parameter a different relative weight or strength allocated to it, and also a linear or not linear, logarhythmic, or parabolic abnormality inflection.

Some laboratory or other parameters can be direct or inverse, meaning the higher the better, like the level of some vitamins, or the higher the worse like TSH or the diabetes marker, the Hemoglobin A1c. In order not to inappropriately affect the final indicator, the direct or inverse characteristic of the parameters needs to be adjusted for.

In another example of a possible setup of indicator ranges, the indicator can also be set up similar to the way blood test results are reported, where results that are too high or too low are abnormal, leaving a middle range of various concentric ranges of values to be acceptable, desirable, optimal or excellent.

In another example, the indicator is constructed from different parameters that could be from different categories or field of science. For example the parameters chosen can be physical measurements for example body weight, laboratory test result values, for example cholesterol, indexes, for example Body Mass Index, quantified results of a questionnaire, for example a score from a depression questionnaire, or an evaluation, for example an evaluation score or a note given by a jury. Any parameter that can be given a number in any way can be used to customize a particular indicator.

The indicator can be used for risk stratification and evaluation of general health as well as subsets of health care related fields including but not limited to: a depression indicator, a fatigue indicator, a cardiovascular indicator, a fitness indicator, a diabetes indicator, an aging indicator, an exhaustion indicator, as well as any indicator one can imagine and design out of two or more quantified and specific, measurable or just given or self-determined parameter or value related to health care or any other fields.

The parameters contributing to the construction of the indicator as well as the various assigned descriptors like weight, contribution, normal ranges, abnormality points and other such variables can all be flexible and able to be tuned to various specific medical conditions and specific desired outcomes or targets.

This indicator can be used primarily in health care and related fields; however other humanistic or scientific fields can use a customized version of this indicator.

Novel and customized versions of this indicator can show in one simple indicator number complex properties and characteristics of materials, people, behaviors, etc.

Indicators can be used to predict future parameters, behaviors, risk factors, and others. Some examples of possible fields of utilization of our indicator include, but are not limited to characterization of nanoparticles, characterization of meat suitability for consumption, suitability for a certain candidate for a specific position, characterization of a propensity of a population to act in a specific way, etc.

What is claimed is:

1. A customizable indicator, based on a mathematical processing of two or more quantified parameters of a system, describing in one simple value, complex properties and characteristics of the system.

2. The indicator from claim 1 wherein parameters properties, including, but not limited to normal range, contribution and abnormality points, can be adjusted, making the indicator able to be customized to specific conditions, purposes and desired outcomes or targets.

3. The indicator in claim 2 wherein the indicator is used primarily in health care and related fields including risk evaluation and stratification.

4. The indicator from claim 2 wherein the indicator is used in any kind of human endeavor including but not limited to humanistic or scientific fields, commerce, trade, management, insurance, or other purposes.

5. The indicator from claim 2 wherein the indicator is used to describe in one simple number, complex properties and characteristics of various systems including but not limited to materials, people, behaviors, and others.

6. The indicator from claim 2 wherein the indicator is used to predict future characteristics including but not limited to behaviors, risk factors, and others.

7. The indicator in claim 3 wherein the indicator is derived from taking two or more laboratory test results as parameters.

8. The indicator in claim 7 wherein the indicator is derived from taking five different laboratory blood test results from the same person, where the indicator value can be between 20 and 500 and where a lower indicator value is better, and an indicator value of less than 100 is considered normal and more than 100 is considered increasingly more abnormal the higher the number is.

9. The indicator in claim 2 wherein the indicator is derived from a combination of parameters that may include, but are generally different than laboratory blood tests, parameters including but not limited to physical measurements, indexes, calculations, ratios, quantified results of a questionnaire, an evaluation or self-evaluation.

10. The indicator in claim 9 wherein the indicator is used for risk stratification and evaluation of a condition, including a medical condition.

11. The indicator in claim 10 wherein the indicator is used for evaluation of personal or collective health, including applications in subsets of health care related fields including but not limited to: a depression indicator, a fatigue indicator, a cardiovascular indicator, a fitness indicator, a diabetes indicator, an aging indicator, an exhaustion indicator, as well as any indicator one can imagine and design out of two or more quantified and specific, measurable or just arbitrarily assigned parameters that relates to health care or other fields.

12. The indicator in claim 2 wherein the indicator is derived from one or more ratios comprising one or more parameters on the numerator and/or one or more parameters on the denominator.

13. The indicator in claim 2 wherein the indicator is used as a prognostic indicator.

14. The indicator in claim 2 wherein the indicator is represented by a range of numbers, or descriptors like letters or other ways of classifying information.
